(19) United States
(12) Patent Application Publication
(10) Pub. No.: US 2026/0335005 A1
(43) Pub. Date: Oct. 29, 2026

(54) INTEGRATED TRANSSEPTAL CROSSING AND THERAPY DELIVERY WIRE

(71) Applicant: Boston Scientific Scimed, Inc., Maple Grove, MN (US)

(72) Inventors: Elliot Wook Lee, Tokyo (JP); Eduardo Moriyama, Richmond (CA); Alison Henstock, Milton (CA); Allan Charles Shuros, St. Paul, MN (US)

(21) Appl. No.: 19/657,518

(22) Filed: Apr. 24, 2026

Related U.S. Application Data
(60) Provisional application No. 63/794,248, filed on Apr. 24, 2025.

(51) Int. Cl. A61B 18/14; A61B 18/00

(52) U.S. Cl. CPC A61B 18/1492; A61B 2018/00083; A61B 2018/00184; A61B 2018/00351; A61B 2018/144; A61B 2018/1475

(57) ABSTRACT

An assembly for a cardiac procedure including a partially insulated wire having a body length, a first end portion terminating in a functional tip and a second end portion terminating in an exposed end. A tubular dilator includes an elongated dilator body having a body length, a proximal end portion and an opposite distal end portion terminating in a distal tip, and a dilator lumen extending through the body and being dimensioned to slidingly receive the wire. A sheath includes a sheath lumen for receiving the dilator for use therewith during a first portion of the cardiac procedure. A secondary device includes a secondary sheath and a secondary therapy catheter for delivery of therapy to a heart during a second portion of the procedure.

### CROSS REFERENCE TO RELATED APPLICATIONS

The present application claims priority to U.S. Provisional Patent Application No. 63/794,248 filed Apr. 24, 2025, the entire disclosure of which is incorporated by reference herein.

### TECHNICAL FIELD

The present disclosure relates to medical systems and methods for accessing a tissue within a heart of a patient. More specifically, the present disclosure relates to medical systems and methods for transseptal crossing and subsequent therapy delivery within a heart of a patient involving a more streamlined and efficient workflow.

### BACKGROUND

Transseptal crossing procedures are essential for accessing the left atrium of the heart during various cardiac interventions. This medical technique involves navigating specialized medical devices through the venous system and crossing the interatrial septum to reach the left atrium. The procedure typically requires precise control and visualization to safely puncture the septum while avoiding damage to surrounding cardiac structures. Furthermore, the procedure typically involves multiple devices and exchanges of such devices, which decreases efficiency of the procedure and may create a disorganized workflow when the devices are exchanged.

Traditional approaches to left atrial access involve the use of specialized puncture devices, followed by exchanging these devices for therapeutic tools. Medical procedures requiring left atrial access include various interventional treatments, with ablation procedures being one common application. Ablation techniques, such as radiofrequency (RF) ablation and cryoablation, are widely used therapeutic approaches that require precise positioning of treatment devices within the left atrium to effectively target cardiac tissue.

Against this background, there exists a continuing need in the industry to provide improved transeptal crossing and secondary therapeutic devices and methods. An object of the present invention is therefore to provide such an apparatus.

### SUMMARY

In Example 1, an assembly for a cardiac procedure includes a wire having a body length, a first end portion terminating in a functional tip and a second end portion terminating in an exposed end. The assembly also includes a tubular dilator including an elongated dilator body having a body length, a proximal end portion and an opposite distal end portion terminating in a distal tip, and a dilator lumen extending through the body and being dimensioned to slidingly receive the wire. The assembly also includes a sheath having a sheath lumen for receiving the dilator for use therewith during a first portion of the cardiac procedure. The assembly also includes a secondary device having a secondary sheath and a secondary therapy catheter for delivery of therapy to a heart during a second portion of the procedure.

Example 2 is the assembly of Example 1 further comprising an energy delivery device that is operable to deliver energy for puncturing tissue, wherein the energy delivery device is connected to the exposed end of the second end portion of the wire.

Example 3 is the assembly of Example 2 wherein the functional tip includes an electrode, and wherein the energy delivery device is configured to deliver radiofrequency energy to the electrode for puncturing tissue.

Example 4 is the assembly of Example 1 wherein the first end portion of the wire aids in puncturing tissue during cardiac procedure, and wherein, after puncturing tissue, the wire can be retracted from the assembly and re-inserted into the assembly by the second end portion of the wire.

Example 5 is the assembly of Example 4 wherein the second end portion of the wire aids in delivery of the secondary device.

Example 6 is the assembly of Example 1 wherein the wire includes a metal core, wherein the wire is insulated along its body length, and wherein the wire is not insulated at least at the functional tip of the first end portion and the exposed end of the second end portion.

Example 7 is the assembly of Example 1 wherein the second end portion of the wire includes a shape that will not puncture tissue.

Example 8 is the assembly of Example 7 wherein the second end portion of the wire includes a curved atraumatic shaped tip.

Example 9 is the assembly of Example 1 wherein the first end portion of the wire includes a J-shape, and wherein the first end portion of the wire includes a pigtail shape.

Example 10 is the assembly of Example 1 wherein the first end portion of the wire is more flexible than the second end portion of the wire.

Example 11 is the assembly of Example 1 wherein the first end portion and the second end portion of the wire are configured to be detectible on an electroanatomical mapping system.

Example 12 is the assembly of Example 4 wherein retracting and re-inserting the wire comprises transitioning from using the first end portion of the wire for transseptal puncture to using the second end portion of the wire for therapy delivery to a heart.

Example 13 is the assembly of Example 12 wherein once rotated to the second end portion of the wire, the wire is configured to maintain left atrial access during transition between transseptal puncture and therapy delivery without requiring a device exchange.

Example 14 is the assembly of Example 1 wherein the first portion of the procedure includes the first end portion of the wire crossing an atrial septum of a heart so that left atrium access is obtained, and wherein the second portion of the procedure includes retracting the wire and re-inserting the wire by the second end portion of the wire so that the wire acts guidewire for insertion of the secondary therapy catheter.

Example 15 is the assembly of Example 1 wherein the wire is configured to streamline workflow of the cardiac procedure by reducing the number of devices exchanged during transition from transseptal access to therapy delivery of the cardiac procedure.

In Example 16, an assembly for a cardiac procedure includes a wire having a body length, a first end portion terminating in a functional tip and a second end portion terminating in an exposed end. The assembly also includes a tubular dilator comprising an elongated dilator body having a body length, a proximal end portion and an opposite distal end portion terminating in a distal tip, and a dilator lumen extending through the body and being dimensioned to slidingly receive the wire. The assembly also includes a sheath having a sheath lumen for receiving the dilator for use therewith during a first portion of the cardiac procedure. The assembly also includes a secondary device having a secondary sheath and a secondary therapy catheter for delivery of therapy to a heart during a second portion of the procedure.

Example 17 is the assembly of Example 16 further comprising an energy delivery device that is operable to deliver energy for puncturing tissue, wherein the energy delivery device is connected to the exposed end of the second end portion of the wire.

Example 18 is the assembly of Example 17 wherein the functional tip includes an electrode, and wherein the energy delivery device is configured to deliver radiofrequency energy to the electrode for puncturing tissue.

Example 19 is the assembly of Example 16 wherein the first end portion of the wire aids in puncturing tissue during cardiac procedure, and wherein, after puncturing tissue, the wire can be retracted from the assembly and re-inserted into the assembly by the second end portion of the wire.

Example 20 is the assembly of Example 16 wherein the wire includes a metal core, wherein the wire is insulated along its body length, and wherein the wire is not insulated at least at the functional tip of the first end portion and the exposed end of the second end portion.

Example 21 is the assembly of Example 16 wherein the second end portion of the wire includes a curved atraumatic shaped tip, and wherein the second end portion comprises a Rosen-shaped tip configured for atraumatic navigation during therapy device delivery.

Example 22 is the assembly of Example 16 wherein the first end portion of the wire includes a J-shape, and wherein the first end portion of the wire includes a pigtail shape.

Example 23 is the assembly of Example 16 wherein the first end portion of the wire is more flexible than the second end portion of the wire.

Example 24 is the assembly of Example 19 wherein retracting and re-inserting the wire comprises transitioning from using the first end portion of the wire for transseptal puncture to using the second end portion of the wire for therapy delivery to a heart.

Example 25 is the assembly of Example 24 wherein once rotated to the second end portion of the wire, the wire is configured to maintain left atrial access during transition between transseptal puncture and therapy delivery without requiring a device exchange.

Example 26 is the assembly of Example 16 wherein the first portion of the procedure includes the first end portion of the wire crossing an atrial septum of a heart so that left atrium access is obtained, and wherein the second portion of the procedure includes retracting the wire and re-inserting the wire by the second end portion of the wire so that the wire acts guidewire for insertion of the secondary therapy catheter.

Example 27 is the assembly of Example 16 wherein the wire is configured to streamline workflow of the cardiac procedure by reducing the number of devices exchanged during transition from transseptal access to therapy delivery of the cardiac procedure.

In Example 28, an assembly for delivering a therapy device into a left atrium of a heart includes a wire having a proximal portion and an opposite distal portion, a flexible coil tube having a functional distal tip and a moveable metal core; wherein the coil tube is dimensioned to slidingly receive the moveable core. The assembly also includes a tubular dilator comprising an elongated dilator body having a body length, a proximal end portion and an opposite distal end portion terminating in a distal tip, and a dilator lumen extending through the body and being dimensioned to slidingly receive the wire. The assembly also includes a sheath for receiving the dilator and the wire for use therewith during a portion of the procedure. The assembly also includes a secondary device having a secondary sheath and a secondary therapy catheter for delivery of therapy to a heart during a portion of the procedure.

Example 29 is the assembly of Example 28 wherein the moveable core includes an exposed proximal end.

Example 30 is the assembly of Example 28 further comprising an energy delivery device that is operable to deliver energy for puncturing tissue, wherein the energy delivery device is connected to the exposed distal section of the moveable core.

Example 31 is the assembly of Example 30 wherein when activated the energy delivery device is configured to deliver radiofrequency energy to the functional distal tip of the coil wire for puncturing tissue.

Example 32 is the assembly of Example 28 wherein the moveable core is configured to be selectively positioned between a first extended position where the functional distal tip is straightened for puncture of tissue and a second retracted position where the functional distal tip forms an atraumatic curved shape.

Example 33 is the assembly of Example 32 wherein the second retracted position includes the moveable core being partially retracted from the distal end portion of the wire, and wherein the second retracted position includes the coil tube assuming a pre-formed atraumatic shape.

Example 34 is the assembly of Example 28 wherein the moveable core is a metal core.

In Example 35 a method for delivering a therapy device into a left atrium of a heart includes providing an at least partially insulated wire having a body length, a first end portion terminating in a functional tip and a second end portion terminating in an exposed end. The method further includes advancing a tubular dilator comprising an elongated dilator body having a body length, a proximal end portion and an opposite distal end portion terminating in a distal tip, and a dilator lumen extending through the body and being dimensioned to slidingly receive the wire. The method further includes advancing a sheath for receiving the dilator and the wire for use therewith. The method further includes advancing the first end portion of the wire proximate a tissue of the heart and puncturing the tissue of the heart using the functional tip of the first end portion of the wire. The method further includes retracting the first end portion of the wire and the dilator. The method further includes rotating the wire to re-insert and advance the second end portion of the wire into the sheath; wherein the second end portion of the wire is distal to the sheath. The method further includes retracting the sheath while maintaining access to the heart through the second end portion of the wire. The method further includes advancing a secondary device having a secondary sheath and a secondary therapy catheter for delivery of therapy to the heart through the second end portion of the wire.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, in accordance with embodiments of the present disclosure.

FIGS. 2A-2F are schematic illustrations of an assembly for transseptal puncture and delivery of a therapy device into a heart, according to embodiments of the present disclosure.

FIGS. 3-4 are schematic illustrations of exemplary integrated wires combining transseptal puncture capability and therapy guidewire functionality of the assembly illustrated in FIGS. 2A-2F, according to embodiments of the present disclosure.

FIGS. 5A-5B are schematic illustrations of another exemplary integrated wire combining transseptal puncture capability and therapy guidewire functionality of the assembly illustrated in FIGS. 2A-2F, according to embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

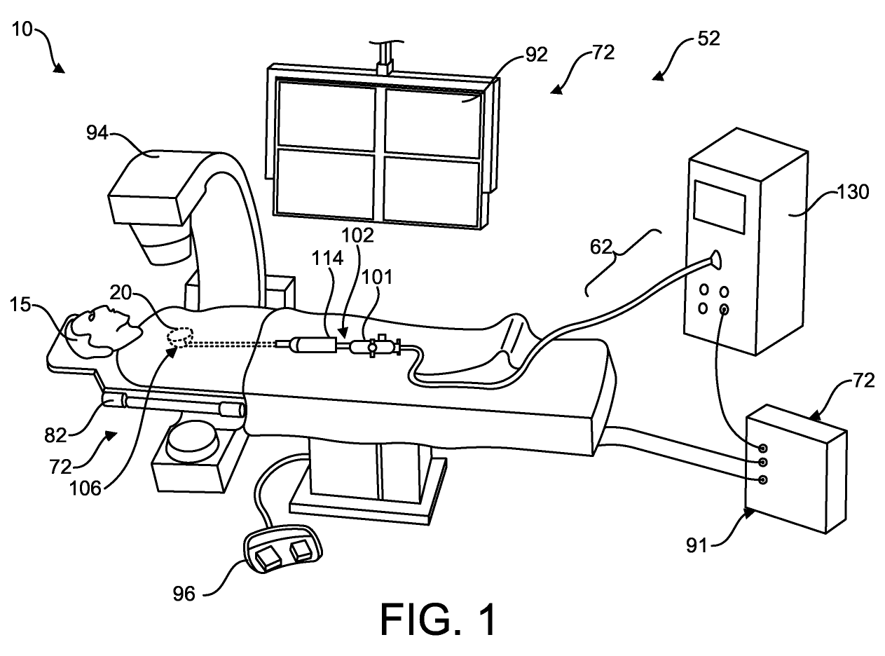

FIG. 1 is a diagram illustrating an exemplary clinical setting 10 for treating a patient 15, and for treating a heart 20 of the patient 15, using an electrophysiology system 52, in accordance with embodiments of the subject matter of the disclosure. The electrophysiology system 52 includes an electroporation catheter system 62 and an electro-anatomical mapping (EAM) system 72, which includes a localization field generator 82, a mapping and navigation controller 91, and a display 92. Also, the clinical setting 10 includes additional equipment such as imaging equipment 94 (represented by the C-arm) and various controller elements, such as a foot controller 96, configured to allow an operator to control various aspects of the electrophysiology system 52. As will be appreciated by the skilled artisan, the clinical setting 10 may have other components and arrangements of components that are not shown in FIG. 1.

The electroporation catheter system 62 includes a catheter 101 having a proximal portion 102 and a distal portion 106, an introducer sheath 114, and an electroporation console 130. Additionally, the electroporation catheter system 62 includes various connecting elements, e.g., cables, umbilical, and the like, that operate to functionally connect the components of the electroporation catheter system 62 to one another and to the components of the EAM system 72. This arrangement of connecting elements is not of critical importance to the present disclosure, and the skilled artisan will recognize that the various components described herein can be interconnected in a variety of ways.

In some embodiments, the introducer sheath 114 is operable to provide a delivery conduit through which the electroporation catheter 101, in particular all or part of the distal portion 106 thereof, can be deployed to the specific target sites within the patient’s heart 20.

In some embodiments, the electroporation catheter system 62 is configured to deliver electric field energy to targeted tissue in the patient’s heart 20 to create tissue apoptosis, rendering the tissue incapable of conducting electrical signals.

The electroporation console 130 is configured to control functional aspects of the electroporation catheter system 62. In embodiments, the electroporation console 130 includes one or more controllers, microprocessors, and/or computers that execute code out of memory to control and/or perform the functional aspects of the electroporation catheter system 62. In embodiments, the memory can be part of the one or more controllers, microprocessors, and/or computers, and/or part of memory capacity accessible through a network, such as the world wide web. In embodiments, the electroporation console 130 includes pulse generator hardware, software and/or firmware configure to generate electrical pulses in predefined waveforms, which are transmitted to electrodes on the electroporation catheter 101 to generate electric fields sufficient to achieve the desired clinical effect, in particular ablation of target tissue through irreversible electroporation. In embodiments, the electroporation console 130 can deliver the pulsed waveforms to the catheter 101 in a monopolar or bipolar mode of operation.

The EAM system 72 is operable to track the location of the various functional components of the electroporation catheter system 62, and to generate high-fidelity three-dimensional anatomical and electro-anatomical maps of the cardiac chambers of interest. In embodiments, the EAM system 72 can be the RHYTHMIA™ HDx mapping system marketed by Boston Scientific Corporation. Also, in embodiments, the mapping and navigation controller 91 of the EAM system 72 includes one or more controllers, microprocessors, and/or computers that execute code out of memory to control and/or perform functional aspects of the EAM system 72, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 52 shown in FIG. 1 is intended to provide a general overview of the various components of the system 52 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 52.

The EAM system 72 generates a localization field, via the field generator 82, to define a localization volume about the heart 20, and one or more location sensors or sensing elements on the tracked device(s), e.g., the electroporation catheter 101, generate an output that can be processed by the mapping and navigation controller 91 to track the location of the sensor, and consequently, the corresponding device, within the localization volume. In the illustrated embodiment, the device tracking is accomplished using magnetic tracking techniques, whereby the field generator 82 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 91 to track the location of the various location sensing electrodes within the localization volume.

In some embodiments, the EAM system 72 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system 72 utilizes the location information for the various tracked devices, along with cardiac electrical activity acquired by, for example, the electroporation catheter 101 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 72 can generate a graphical representation of the various tracked devices within the geometric anatomical map and/or the electro-anatomical map.

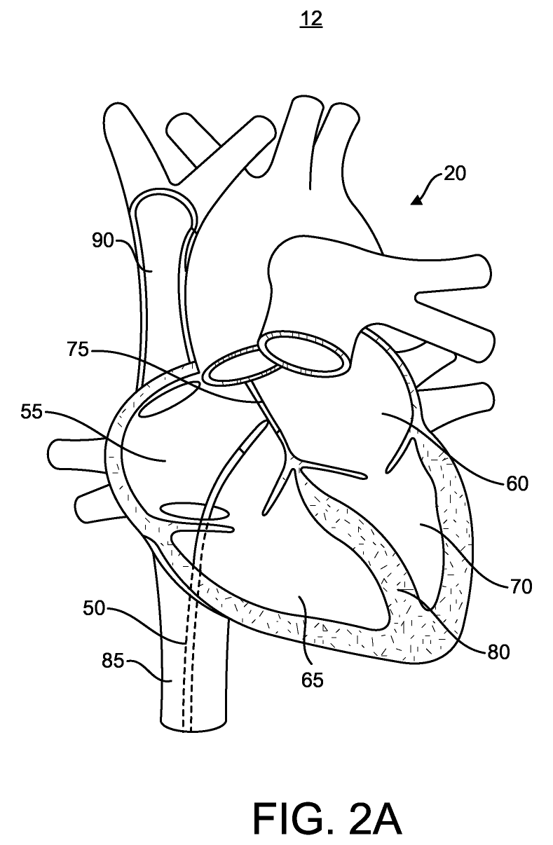

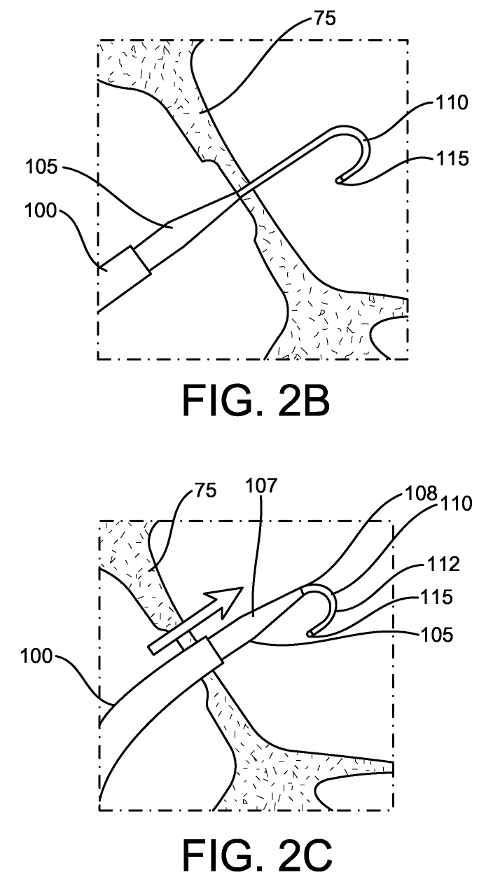

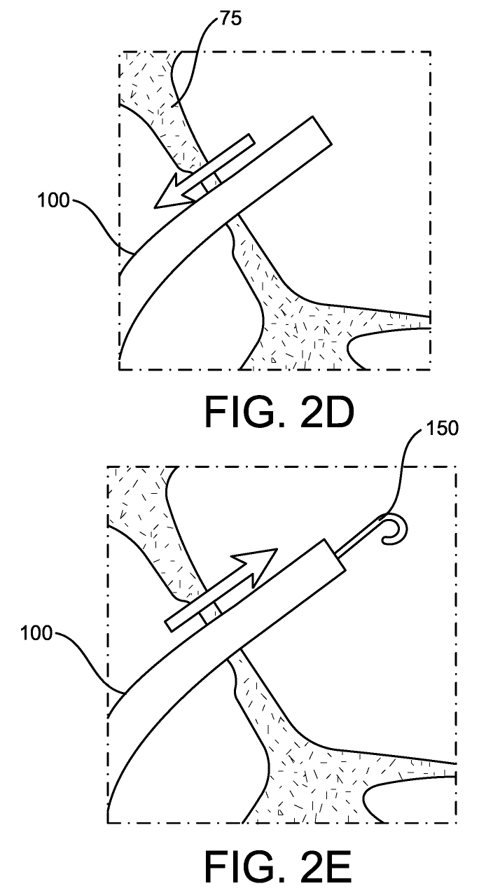

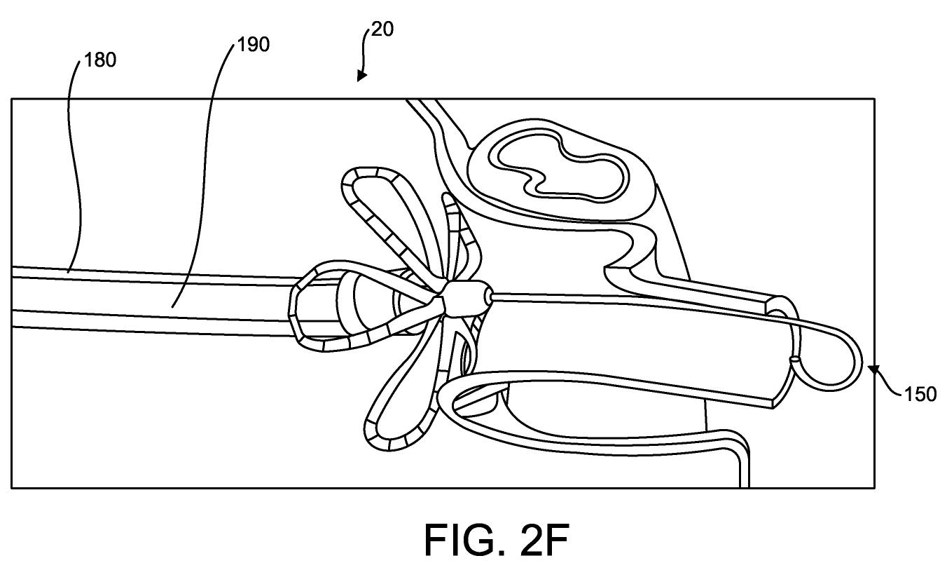

In an embodiment, the exemplary clinical setting 10 may be used for a medical procedure within a patient’s heart for gaining transseptal access and subsequent delivery of secondary therapeutic devices. FIGS. 2A-2F are schematic illustrations of a medical procedure 12 within a patient’s heart 20 utilizing a transseptal access system 50 and subsequent deployment of therapeutic devices according to embodiments of the disclosure. In an embodiment, as will be discussed in greater detail below, an integrated device is disclosed which combines multiple functionalities for cardiac procedures, as illustrated in the exemplary clinical setting 10 for treating a patient 15 using an electrophysiology system 52.

As is known, the human heart 20 has four chambers, a right atrium 55, a left atrium 60, a right ventricle 65 and a left ventricle 70. Separating the right atrium 55 and the left atrium 60 is an atrial septum 75, and separating the right ventricle 65 and the left ventricle 70 is a ventricular septum 80. As is further known, deoxygenated blood from the patient’s body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 12, illustrated in FIGS. 2A-2F, is an exemplary embodiment for providing access to the left atrium 60 using the transseptal access system 50 and subsequent deployment of the aforementioned therapeutic devices within the left atrium 60. As shown in FIGS. 2A-2C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, as illustrated in FIGS. 2B-2C, the transseptal access system 50 includes an introducer sheath 100, a dilator 105 having a dilator body 107 and a tapered distal tip portion 108, and a radiofrequency (RF) perforation device 110 having distal end portion 112 terminating in a tip electrode 115. As shown, in the assembled use state, the RF perforation device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100 and dilator 105 have been positioned in the SVC 90, the guidewire is removed from the body, and the sheath 100 and the dilator 105 are retracted so that their distal ends are positioned in the right atrium 55. The RF perforation device 110 described can then be introduced into the dilator 105, and advanced toward the heart 20. In certain embodiments, the dilator may be introduced into the body without a need for the sheath 100.

Subsequently, the user may position the distal end of the dilator 105 against the atrial septum 75, which can be done under imaging guidance. The RF perforation device 110 is then positioned such that the tip electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation, as illustrated in FIG. 2B. In these embodiments, when the tip electrode 115 has passed through the target tissue, that is, when it has reached the left atrium 60, energy delivery is stopped. In some embodiments, the step of delivering energy occurs over a period of between about 1 second and about 5 seconds. In other embodiments, the perforation device 110 may be a mechanical perforation device, and thus the perforation device 110 may mechanically puncture the target site.

With the tip electrode 115 of the RF perforation device 110 having crossed the atrial septum 75, the dilator 105 can be advanced forward, with the tapered distal tip portion 108 operating to gradually enlarge the perforation to permit advancement of the distal end of the sheath 100 into the left atrium 60, as illustrated in FIG. 2C.

In current cardiac procedures, once the RF perforation device 110 has crossed the atrial septum 75 and the distal end of the sheath 100 is advanced into the left atrium 60 so that left atrium access has been obtained, the puncture device 110 may then be removed, and a guidewire is inserted through the transseptal sheath 100 and/or the dilator 105. The inserted guidewire may then allow for the exchange of the transseptal sheath 100 for a therapy sheath. A therapy catheter can then be introduced over the guidewire for subsequent delivery of therapy to the heart. Other devices and tools may also be used during this exchange. As discussed, current cardiac procedures involve the use of multiple devices and exchanges of such devices, which decreases efficiency of the procedure and may create a disorganized workflow when the devices are exchanged.

The present disclosure describes novel systems and methods for providing safe access and delivery of therapeutic devices to the heart. As will be explained in greater detail herein, the embodiments of the present disclosure improve the effectiveness, safety, efficiency, and streamline the workflow of integrated perforation device-based systems by integrating transseptal puncture capabilities with therapy guidewire functionality for a cardiac procedure.

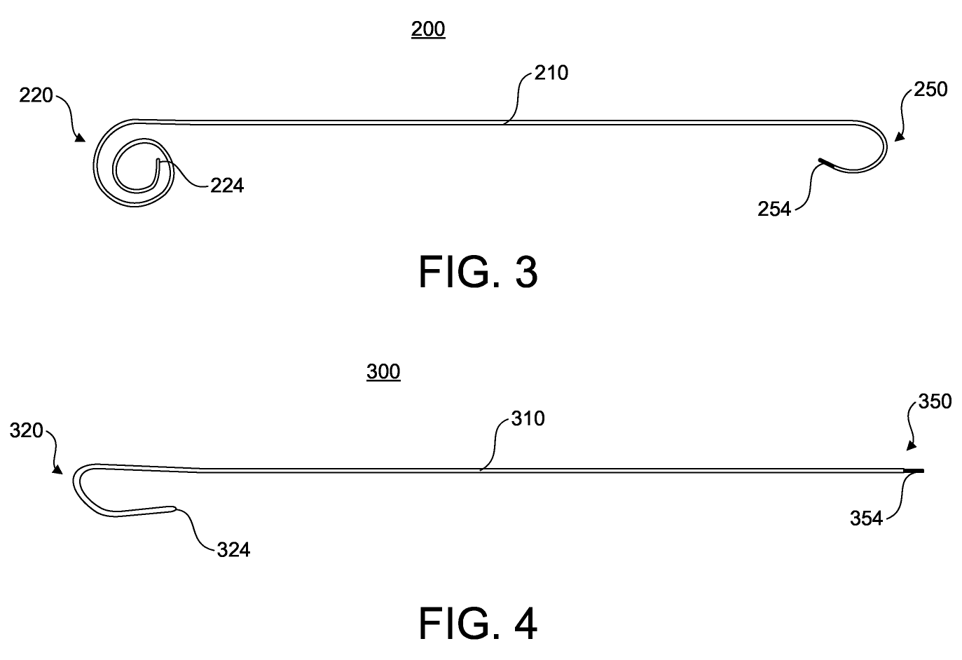

FIGS. 3-4 are illustrations of exemplary integrated wires combining transseptal puncture capability and therapy guidewire functionality within a single wire for cardiac procedures, according to embodiments of the present disclosure. In an embodiment, the integrated wire is an at least partially insulated wire 200, 300 insulated along its body length. The wire 300 of FIG. 4 may be substantially structurally and functionally identical to the wire 200 of FIG. 3, except as described in connection with FIG. 4. The at least partially insulated wire 200, 300 (i.e., the integrated wire having both transseptal puncture capability and therapy guidewire functionality) for cardiac procedure includes a wire body having a body length 210, 310 and distinct functional ends. The wire 200, 300 includes a first end portion 220, 320 terminating in a functional tip 224, 324 and a second end portion 250, 350 terminating in an exposed end 254, 354. In an embodiment, the exposed end 254, 354 includes no insulation on the second end portion 250, 350 of the wire 200, 300.

In an embodiment, an energy delivery device operable to deliver energy for puncturing tissue may be connected to the exposed end 254, 354 of the second end portion 250, 350 of the wire 200, 300. In an embodiment, the functional tip 224, 324 includes an electrode, and the energy delivery device is configured to deliver radiofrequency energy traveling from the exposed end 254, 354 of the second end portion 250, 350 to the electrode of the functional tip 224, 324 of the first end portion 220, 320 for puncturing tissue at a target site, as discussed above and illustrated in FIG. 2B. Thus, in an embodiment, the first end portion 220, 320 may be used for transseptal puncture and crossing to the left atrium of the heart, as discussed above. In an embodiment, the wire 200, 300 incorporates a metal core and maintains insulation along its body length 210, 310, with strategic exposure at both the functional tip 224, 324 of the first end portion 220, 320 and the exposed end 254, 354 of the second end portion 250, 350. In other embodiments, the first end portion 220, 320 may include a mechanical puncture device for mechanically perforating a target tissue, instead of an RF perforation tip.

In an embodiment, the first end portion 220, 320 of the integrated wire 200, 300 may be pre-formed to assume a curved atraumatic pre-determined shape. In an embodiment, as shown, the first end portion 220 of the integrated wire 200 may assume a pigtail shape (as shown in FIG. 3), a J-shape (as shown in FIG. 4), or other shapes selected. In an embodiment, the pre-formed shapes can advantageously function to minimize the risk of unintended contact between the tip electrode 224 and tissue within the left atrium of the heart and can also operate to anchor the first end portion 220 of the wire within the left atrium during subsequent procedural steps, if needed in the procedure. In an embodiment, as illustrated in FIG. 3, while the first end portion includes a pre-formed shape the most distal portion of the first end portion 220 includes a straight end shape. In an embodiment, when the first end portion of the wire 200 projects out of the sheath or dilator, as discussed above, the first end portion is deployed in a straight-shape condition so to apply RF energy via the tip electrode to the target tissue. Once RF energy is applied and the tissue is perforated, the first end portion 220 is the advanced out of the dilator and/or sheath and assumes its pre-formed shape, i.e., a pigtail shape or J-tip shape as illustrated in FIGS. 3 and 4.

In an embodiment, the second end portion 250, 350 of the integrated wire 200, 300 may provide guidewire functionality. In an embodiment, after puncturing a target tissue by the first end portion 220, and while the sheath 100 of FIGS. 2A-2C is still positioned in the left atrium, the wire 200 may be taken out (i.e., pulled out, retracted) from the assembly and re-inserted into the assembly by the second end portion 250 of the wire 200 being placed distally within the assembly. In an embodiment, the second end portion 250 may aid in delivery of a secondary sheath and a secondary catheter for delivery of therapeutic devices within the heart. Thus, in an embodiment, since the second end portion 250 is not puncturing tissue, the distal portion of the second end portion 250 does not include a straight portion, as does the distal end portion of the first end portion 220 does. In an embodiment, the second end portion 250 of the integrated wire 200 may be pre-formed to assume an atraumatic pre-determined shape. In an embodiment, the second end portion 250 of the integrated wire 200 may assume a Rosen tip shape (as shown in FIG. 3), a straight end shape (as shown in FIG. 4), or other shapes known in the art for a guidewire. In an embodiment, when the second end portion 250 is deployed into the heart and the left atrium it may immediately assume its pre-formed shape.

Returning to FIGS. 2A-2F, an assembly for cardiac procedure 12 is disclosed, according to embodiments of the present disclosure. The assembly includes an at least partially insulated wire 200, 300 having a body length 210, 310, a first end portion 220, 320 terminating in a functional tip 224, 324 and a second end portion 250, 350 terminating in an exposed end 254, 354 (see FIGS. 3-4 discussed above). In an embodiment, as illustrated in FIGS. 2B-2C, the assembly further includes a tubular dilator 105 comprising an elongated dilator body 107 having a body length, a proximal end portion and a distal end portion terminating in a distal tip, a hub at the proximal end of the body having a hub length, and a dilator lumen extending through the hub and the body and being dimensioned to slidingly receive the flexible wire 200, 300. In an embodiment, the assembly further includes a sheath 100 having a sheath lumen for receiving the dilator for use therewith during a portion of the cardiac procedure 12. In an embodiment, as will be discussed in greater detail below, the assembly further includes a secondary device having a secondary sheath 180 and a secondary therapy catheter 190 for delivery of therapy to a heart during a portion of the procedure 12. In an embodiment, the transseptal sheath 100 and/or the secondary sheath 180 may be a steerable sheath. In an embodiment, the wire 200, 300 may include a metal core. In an embodiment, the wire 200, 300 is insulated along its body length. In some embodiments, the wire 200, 300 is not insulated at least at the functional tip 224 of the first end portion 220 and the exposed end 254 of the second end portion 250.

In an embodiment, as illustrated in FIG. 2D, once the perforation device 110 (i.e., the first end portion 220, 320 of FIGS. 3-4) has crossed the atrial septum 75 and the distal end of the sheath 100 is advanced into the left atrium 60 so that left atrium access has been obtained, with the transseptal sheath 100 holding access, the at least partially insulated wire 200, 300 may be retracted from the heart. Thus, in an embodiment, the first end portion 220 is retracted from the heart through the sheath 100. In an embodiment, the dilator 105 is also retracted from the assembly. In an embodiment, as illustrated in FIG. 2E, the at least partially insulated wire 200, 300 may then be rotated (i.e., flipped) by the operator so that the second end portion 250, acting as a guidewire, is inserted into the left atrium through the transseptal sheath 100. In an embodiment, rotating the wire 200, 300 may include transitioning from utilizing the first end portion 220 for transseptal puncture to utilizing the second end portion 250 for therapy delivery to the heart. In an embodiment, the at least partially insulated wire 200, 300 may then maintain left atrium access as the transseptal sheath 100 is retracted and exchanged for the secondary sheath 180. In an embodiment, the secondary sheath 180 may then provide support for the insertion of the secondary therapy catheter 190, as illustrated in FIG. 2F. Thus, in an embodiment, the second end portion 250, 350 of the wire 200, 300 aids in delivery of the secondary device into the heart of the patient.

In an embodiment, a first portion of the cardiac procedure 12 may involve the first end portion 220, 320 of the at least partially insulated wire 200, 300 crossing the atrial septum 75 so that left atrium access has been obtained. In an embodiment, a second portion of the procedure 12 may include retracting the at least partially insulated wire 200, 300 and reinserting the wire 200, 300 by the second end portion 250, 350 of the wire 200, 300 to then be able to insert a secondary sheath 180 and therapy catheter 190 for delivery of therapy to the heart. Thus, in an embodiment, the at least partially insulated wire 200, 300 may include both transseptal puncture and therapy guidewire functionality so to reduce the number of devices required in a transseptal cardiac procedure.

In some instances of the embodiments described herein above, the integrated wire 200, 300 may be use in connection with the VersaCross™ steerable sheath or the SupraCross™ steerable sheath (all by Baylis Medical Company Inc.). In some embodiments, the RF guidewire may be the PowerWire™ RF guidewire (Baylis Medical Company Inc.). In other embodiments, the secondary therapy catheter may be the FARAWAVE™ catheter from the FARAPULSE™ products (Baylis Medical Company Inc.). In other embodiments, the secondary sheath may be the FARADRIVE™ steerable sheath from the FARAPULSE™ products (Baylis Medical Company Inc.). Alternatively, other guidewires, sheaths and catheters maybe used for embodiments of methods and/or procedures as described herein above.

In an embodiment, the wire 200 incorporates a metal core and maintains insulation along a portion of its body length 210. In certain embodiments, one exemplary class of materials for construction of the insulated portion can include various grades of polytetrafluoroethylene (PTFE), polyetheretherketone (PEEK), Polyimide, among others. In some embodiments, one exemplary class of materials for construction of the metal core of the wire 200 can include various grades of stainless steel, nitinol, tungsten, other medical-grade metal alloys, among others. In an embodiment, the exposed end 254 is electrically conductive and is capable of transferring radiofrequency energy supplied by an external RF generator to the functional tip 224 for subsequent delivery to the target tissue in a transseptal crossing procedure, as described above. Any biocompatible electrically conductive material may be selected for construction of the exposed end 254 of the second end portion 250. Exemplary materials may include stainless steel, nickel-titanium alloy, and the like. In an embodiment, the functional tip 224 may include a platinum electrode.

In an embodiment, the diameter of the first end portion 220 may be the same as the diameter of the second end portion 250. In an embodiment, the diameter of the first end portion 220 and the second end portion 250 is between 0.020 inches and 0.050 inches. In an embodiment, the diameter of the first end portion 220 and the second end portion 250 is between 0.027 inches and 0.040 inches. In an embodiment, the diameter of the first end portion 220 and the second end portion 250 is between 0.032 inches and 0.035 inches. In other embodiments, the diameter of the first end portion 220 may be different than the diameter of the second end portion 250. In an embodiment, the first end portion 220 and the second end portion 250 of the wire 200 are configured to be detectible on an electroanatomical mapping system 72 of FIG. 1.

In an embodiment, the length of the integrated wire 200, 300 is between 160 cm to 210 cm. In some embodiments, the length of the integrated wire 200, 300 is between 170 cm to 200 cm. In other embodiments, the length of the integrated wire 200, 300 is between 175 cm to 185 cm. In some embodiments, the stiffness of the first end portion 220 and the second end portion 250 may vary depending on use. In an embodiment, the first end portion 220 may be more flexible than the second end portion 250. In an embodiment, for example, the first end portion 220 has a first end portion stiffness or rigidity with a flexural rigidity of between 50 GPa and 80 GPa. In an embodiment, for example, the first end portion 220 has a first end portion stiffness or rigidity with a flexural rigidity of between 60 GPa and 70 GPa. In an embodiment, for example, the first end portion 220 has a first end portion stiffness or rigidity with a flexural rigidity of between 63 GPa and 83 GPa. In an embodiment, for example, the first end portion 220 has a first end portion stiffness or rigidity with a flexural rigidity of less than about 65.4 GPa. In an embodiment, the second end portion 250 has a second end portion stiffness or rigidity with a flexural rigidity of between 1 GPa and 30 GPa. In an embodiment, the second end portion 250 has a second end portion stiffness or rigidity with a flexural rigidity of between 5 GPa and 20 GPa. In an embodiment, the second end portion 250 has a second end portion stiffness or rigidity with a flexural rigidity of between 10 GPa and 15 GPa.

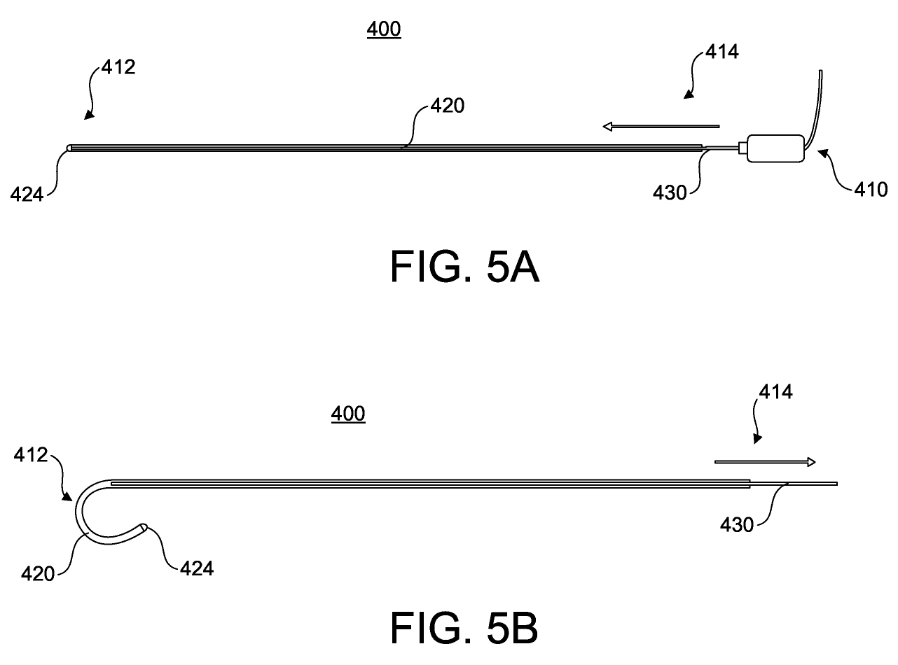

FIGS. 5A-5B illustrate another exemplary integrated wire 400 combining transseptal puncture capability and therapy guidewire functionality within a single wire for cardiac procedures. In an embodiment, an assembly for delivering a therapy device into a left atrium of a heart is disclosed. The assembly includes a wire 400 having a proximal portion 414 and an opposite distal portion 412, a flexible coil tube 420 having a functional distal tip 424 and a moveable metal core 430. In an embodiment, the coil tube 420 is dimensioned to slidingly receive the moveable core 430. In an embodiment, the moveable core 430 can transition between an extended position (see FIG. 5A) for RF puncture of a target tissue, as discussed above in FIGS. 2B-2C, and a retracted position (see FIG. 5B) forming a J-tip for guidewire functionality, as discussed above in FIGS. 2D-2F, for delivery of a therapy catheter to the heart.

In an embodiment, the moveable core 430 is a metal core. In some embodiments, one exemplary class of materials for construction of the moveable metal core 430 of the wire 400 can include various grades of stainless steel, nitinol, tungsten, other medical-grade metal alloys, among others. In an embodiment, the flexible coil tube 420 is an insulated flexible tube which slidingly receives the moveable core 430. In certain embodiments, one exemplary class of materials for construction of the insulated flexible coil tube 420 can include various grades of polytetrafluoroethylene (PTFE), polyetheretherketone (PEEK), Polyimide, among others. In an embodiment, since the coil tube 420 is dimensioned to slidingly receive the moveable core 420, a majority of the metal core 430 may be covered by the flexible coil tube 420. However, in an embodiment, the moveable core 430 includes an exposed proximal end. In an embodiment, an energy delivery device 410 operable to deliver energy for perforating tissue may be connected to the exposed proximal end of the moveable metal core 430 of the wire 400, as show in FIG. 5A. In an embodiment, the functional distal tip 424 includes an electrode, and the energy delivery device 410 is configured to deliver radiofrequency energy traveling from the proximal exposed end of the moveable metal core 430 to the electrode of the functional tip 424 of the flexible coil tube 420 to perforate tissue at a target site, as discussed above and illustrated in FIG. 2B. Thus, in an embodiment, the extended position of the wire 400 may be inserted into a sheath 100 and dilator 105 and used for transseptal puncture and crossing to the left atrium of the heart, as discussed above in FIGS. 2B-2C.

In an embodiment, once the wire 400, specifically the functional distal tip 424, has perforated the target tissue the energy delivery device may be disconnected from the integrated wire 400. In an embodiment, once the wire 400 has crossed the atrial septum 75 and the distal end of the sheath 100 is advanced into the left atrium 60 so that left atrium access has been obtained, the moveable metal core 430 may be partially retracted from the flexible coil tube 420 so that the wire assumes the retracted position, as show in FIG. 5B. In some embodiments, in a retracted position, the distal portion of the flexible coil tube 420 may assume an atraumatic J-shape configuration. In some embodiments, the atraumatic J-shape configuration of the distal portion of the wire 400 is created because the metal core 430 is partially retracted from the flexible coil tube 420, allowing the coil tube 420 to assume its pre-formed J-shape configuration. In an embodiment, the retracted position of the wire 400 acts as a guidewire for the assembly discussed above. In an embodiment, once the metal core 430 is partially retracted and the coil tube 420 assumes its pre-formed shape configuration so to be atraumatic and not be able to puncture tissue, the dilator 105 and the transseptal sheath 100 may be retracted from the assembly. In an embodiment, with the wire 400 acting as the guidewire and maintaining left atrium access, a secondary sheath 180 and a secondary therapy catheter 190 may be inserted, as discussed and illustrated in FIG. 2F. Thus, in an embodiment, the retracted position of the wire 400 aids in delivery of the secondary therapy device into the heart of the patient. Thus, in an embodiment, the integrated wire 400 may include both transseptal puncture capabilities and therapy guidewire functionality for a cardiac procedure. In some embodiments, the wire 400 is configured to streamline workflow by reducing the number of devices needed during transition from transseptal access to therapy delivery.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An assembly for a cardiac procedure, the assembly comprising:
a wire having a body length, a first end portion terminating in a functional tip and a second end portion terminating in an exposed end;
a tubular dilator comprising an elongated dilator body having a body length, a proximal end portion and an opposite distal end portion terminating in a distal tip, and a dilator lumen extending through the body and being dimensioned to slidingly receive the wire;
a sheath having a sheath lumen for receiving the dilator for use therewith during a first portion of the cardiac procedure; and
a secondary device having a secondary sheath and a secondary therapy catheter for delivery of therapy to a heart during a second portion of the procedure.

2. The assembly of claim 1, further comprising an energy delivery device that is operable to deliver energy for puncturing tissue, wherein the energy delivery device is connected to the exposed end of the second end portion of the wire.

3. The assembly of claim 2, wherein the functional tip includes an electrode, and wherein the energy delivery device is configured to deliver radiofrequency energy to the electrode for puncturing tissue.

4. The assembly of claim 1, wherein the first end portion of the wire aids in puncturing tissue during cardiac procedure, and wherein, after puncturing tissue, the wire can be retracted from the assembly and re-inserted into the assembly by the second end portion of the wire.

5. The assembly of claim 1, wherein the wire includes a metal core, wherein the wire is insulated along its body length, and wherein the wire is not insulated at least at the functional tip of the first end portion and the exposed end of the second end portion.

6. The assembly of claim 1, wherein the second end portion of the wire includes a curved atraumatic shaped tip, and wherein the second end portion comprises a Rosen-shaped tip configured for atraumatic navigation during therapy device delivery.

7. The assembly of claim 1, wherein the first end portion of the wire includes a J-shape, and wherein the first end portion of the wire includes a pigtail shape.

8. The assembly of claim 1, wherein the first end portion of the wire is more flexible than the second end portion of the wire.

9. The assembly of claim 4, wherein retracting and re-inserting the wire comprises transitioning from using the first end portion of the wire for transseptal puncture to using the second end portion of the wire for therapy delivery to a heart.

10. The assembly of claim 9, wherein once rotated to the second end portion of the wire, the wire is configured to maintain left atrial access during transition between transseptal puncture and therapy delivery without requiring a device exchange.

11. The assembly of claim 1, wherein the first portion of the procedure includes the first end portion of the wire crossing an atrial septum of a heart so that left atrium access is obtained, and wherein the second portion of the procedure includes retracting the wire and re-inserting the wire by the second end portion of the wire so that the wire acts guidewire for insertion of the secondary therapy catheter.

12. The assembly of claim 1, wherein the wire is configured to streamline workflow of the cardiac procedure by reducing the number of devices exchanged during transition from transseptal access to therapy delivery of the cardiac procedure.

13. An assembly for delivering a therapy device into a left atrium of a heart, the assembly comprising:
a wire having a proximal portion and an opposite distal portion, a flexible coil tube having a functional distal tip and a moveable metal core, wherein the coil tube is dimensioned to slidingly receive the moveable core;
a tubular dilator comprising an elongated dilator body having a body length, a proximal end portion and an opposite distal end portion terminating in a distal tip, and a dilator lumen extending through the body and being dimensioned to slidingly receive the wire;
a sheath for receiving the dilator and the wire for use therewith during a portion of the procedure; and
a secondary device having a secondary sheath and a secondary therapy catheter for delivery of therapy to a heart during a portion of the procedure.

14. The assembly of claim 13, wherein the moveable core includes an exposed proximal end.

15. The assembly of claim 13, further comprising an energy delivery device that is operable to deliver energy for puncturing tissue, wherein the energy delivery device is connected to the exposed distal section of the moveable core.

16. The assembly of claim 15, wherein when activated the energy delivery device is configured to deliver radiofrequency energy to the functional distal tip of the coil wire for puncturing tissue.

17. The assembly of claim 13, wherein the moveable core is configured to be selectively positioned between a first extended position where the functional distal tip is straightened for puncture of tissue and a second retracted position where the functional distal tip forms an atraumatic curved shape.

18. The assembly of claim 17, wherein the second retracted position includes the moveable core being partially retracted from the distal end portion of the wire, and wherein the second retracted position includes the coil tube assuming a pre-formed atraumatic shape.

19. The assembly of claim 13, wherein the moveable core is a metal core.

20. A method for delivering a therapy device into a left atrium of a heart, the method comprising:
providing an at least partially insulated wire having a body length, a first end portion terminating in a functional tip and a second end portion terminating in an exposed end;
advancing a tubular dilator comprising an elongated dilator body having a body length, a proximal end portion and an opposite distal end portion terminating in a distal tip, and a dilator lumen extending through the body and being dimensioned to slidingly receive the wire;
advancing a sheath for receiving the dilator and the wire for use therewith;
advancing the first end portion of the wire proximate a tissue of the heart and puncturing the tissue of the heart using the functional tip of the first end portion of the wire;
retracting the first end portion of the wire and the dilator;
rotating the wire to re-insert and advance the second end portion of the wire into the sheath, wherein the second end portion of the wire is distal to the sheath;
retracting the sheath while maintaining access to the heart through the second end portion of the wire; and
advancing a secondary device having a secondary sheath and a secondary therapy catheter for delivery of therapy to the heart through the second end portion of the wire.
